(19) United States
(12) Patent Application Publication
(10) Pub. No.: US 2026/0364384 A1
(43) Pub. Date: Nov. 19, 2026

(54) THERAPUETIC ULTRASOUND SYSTEM USING OVERLAPPING FOCAL VOLUMES

(71) Applicant: West Virginia University Board of Governors on behalf of West Virginia University, Morgantown, WV (US)

(72) Inventors: Ali Rezai, Morgantown, WV (US); Victor Finomore, Morgantown, WV (US); Mark Schafer, Morgantown, WV (US); Geoff Adams, Morgantown, WV (US); Jeff Carpenter, Morgantown, WV (US)

(21) Appl. No.: 19/458,180

(22) Filed: Jan. 23, 2026

Related U.S. Application Data
(60) Provisional application No. 63/748,746, filed on Jan. 23, 2025.

(51) Int. Cl. A61N 7/00

(52) U.S. Cl. CPC A61N 7/00; A61N 2007/0026; A61N 2007/0095

(57) ABSTRACT

Systems and methods are provided for focused ultrasound neuromodulation. A first phased array ultrasound transducer is positioned within a fixation device to provide a first ultrasound beam through the ultrasound temporal window of a patient with a first focal volume within a region of interest. A second phased array ultrasound transducer is positioned within the fixation device to provide a second ultrasound beam through one of a frontal bone, a parietal bone, a sagittal suture, or a coronal suture of the patient with a second focal volume within a region of interest. A signal dose within each of the focal volumes is less than a threshold dose. A system control controls each of the ultrasound transducers such that the focal volumes overlap as to collectively provide a signal dose within a region of overlap in the region of interest that is not less than the threshold dose.

### RELATED APPLICATION

This application claims priority from U.S. Provisional Application No. 63/748,746, filed 23 Jan. 2025, the subject matter of which is incorporated herein by reference in its entirety.

### TECHNICAL FIELD

This disclosure relates generally to the field of medical systems, and more particularly to a therapeutic ultrasound system for neuromodulation.

### BACKGROUND

Neuromodulation refers to an emerging class of medical therapies that target the nervous system for restoration of function, relief of pain, or control of symptoms. The therapies consist primarily of targeted stimulation by various forms of energy, such as electrical stimulation, magnetic stimulation, such as transcutaneous magnetic stimulation, as well as sound, in focused ultrasound systems. Neuromodulation, particularly non-invasive neuromodulation using focused ultrasound, appears promising in treating a number of disorders, but modulating targets deep within the brain poses challenges.

### SUMMARY OF THE INVENTION

In accordance with one aspect of the present invention, a system includes a fixation device, a first phased array ultrasound transducer, and a second phased array ultrasound transducer. The first phased array ultrasound transducer is positioned within the fixation device to provide a first ultrasound beam through the temporal ultrasound region with a first focal volume within a region of interest. A signal dose within the first focal volume is less than a threshold dose associated with therapeutic efficacy. The second phased array ultrasound transducer is positioned within the fixation device to provide a second ultrasound beam through one of a frontal bone, a parietal bone, a sagittal suture, or a coronal suture of the patient with a second focal volume within a region of interest. A signal dose within the second focal volume is less than the threshold dose. A system control controls each of the first phased array ultrasound transducer and the second phased array ultrasound transducer such that the first focal volume overlaps with the second focal volume as to collectively provide a signal dose within a region of overlap in the region of interest that is not less than the threshold dose.

In accordance with another aspect of the invention, a method is provided. A first phased array ultrasound transducer is positioned such that a first ultrasound beam provided by the first phased array ultrasound transducer will pass through the temporal ultrasound window of a patient. A second phased array ultrasound transducer is positioned such that a second ultrasound beam provided by the second phased array ultrasound transducer will pass through one of a frontal bone, a parietal bone, a sagittal suture, or a coronal suture of the patient. The first ultrasound beam is provided with a first focal volume within a region of interest with a signal dose within the first focal volume being less than a threshold dose associated with therapeutic efficacy. The second ultrasound beam is provided with a second focal volume within the region of interest, with a signal dose within the second focal volume being less than the threshold dose. Each of the first ultrasound beam and the second ultrasound beam are controlled such that the first focal volume overlaps with the second focal volume as to collectively provide a signal dose within a region of overlap in the region of interest that is not less than the threshold dose.

In accordance with one aspect of the present invention, a system includes a fixation device, a segmented annular array ultrasound transducer, and a rectangular array ultrasound transducer. The segmented annular array ultrasound transducer is positioned within the fixation device to provide a first ultrasound beam through the temporal ultrasound window with a first focal volume within a region of interest. A signal dose within the first focal volume is less than a threshold dose associated with therapeutic efficacy. The rectangular array ultrasound transducer is positioned within the fixation device to provide a second ultrasound beam through one of the frontal bone, a parietal bone, a sagittal suture, or the coronal suture with a second focal volume within a region of interest. A signal dose within the second focal volume is less than the threshold dose. A system control controls each of the first array ultrasound transducer and the second array ultrasound transducer such that the first focal volume overlaps with the second focal volume as to collectively provide a signal dose within a region of overlap in the region of interest that is not less than the threshold dose.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates a system for providing neuromodulation via focused ultrasound;

FIG. 2A is a diagram illustrating an example placement of the first phased array ultrasound transducer and the second phased array ultrasound transducer of the system of FIG. 1;

FIG. 2B is a diagram illustrating example skull landmarks;

FIG. 3 illustrates a simulation of the region of overlap of the first and second ultrasound beams of FIG. 2 within the region of interest;

FIG. 4 illustrates one example of a segmented annular array that can be used with the system of FIG. 1;

FIG. 5 illustrates one example of a rectangular array transducer, having two rows of elements, that can be used with the system of FIG. 1;

FIG. 6 illustrates another example of a rectangular array transducer, implemented as a segmented rectangular array having a plurality of rectangular sections, that can be used with the system of FIG. 1;

FIG. 7 illustrates a further example of a rectangular array transducer, implemented as a segmented rectangular array having a plurality of rectangular sections, that can be used with the system of FIG. 1;

FIG. 8 illustrates a still further example of a rectangular array transducer, implemented as a curved rectangular array, that can be used with the system of FIG. 1;

FIG. 9 illustrates an example implementation of a wearable fixation device that can be used with the system of FIG. 1;

FIG. 10 illustrates another example implementation of a fixation device that can be used with the system of FIG. 1;

FIG. 11 illustrates an example implementation of a system for providing neuromodulation via focused ultrasound;

FIG. 12 illustrates one example of a method for providing neuromodulation via focused ultrasound; and FIG. 13 is a schematic block diagram illustrating an exemplary system of hardware components capable of implementing examples of the systems and methods disclosed in FIGS. 1-12.

### DETAILED DESCRIPTION

A transducer, as used herein, is a device that produces an ultrasound signal, or beam. Transducers can be implemented in arrays of elements to allow for electrical steering of the ultrasound signal.

A “phased array transducer,” as used herein, is a transducer having a plurality of elements that can be individually controlled using signals of varying phase and amplitude to allow for electronic steering of an ultrasound beam produced by the transducer.

The “temporal ultrasound window” refers to a region around the pterion, the junction of the frontal, parietal, temporal, and sphenoid bones, in which the skull is particularly thin.

A “focal volume” as used herein, is the region of an ultrasound signal in which the intensity is greater than half of the maximum intensity. The focal volume is generally an elongated ellipsoid, region, often referred to as “cigar-shaped,” with a longest axis along the direction of the ultrasound signal and a substantially circular in cross-section along the axis of ultrasound propagation that tapers from a largest diameter at a center of the region to a smallest diameter on each end.

A “dose” of a signal, as used herein, can refer to any characteristic of an ultrasound signal relating to a quantity of energy delivered over one or both of a unit of area or unit of time. In this usage, a signal dose is explicitly intended to include a pressure

imposed by the ultrasound signal, including, but not limited to a duration, a spatial peak pressure of the signal, an intensity of the signal, and a power of the signal.

“Nervous system tissue”, as used herein, refers to axons, neurons, synapses, glial cells, and supporting tissue in the brain and central, peripheral, and autonomic nervous system.

A “skull landmark,” as used herein, refers to a feature of the skull that can be found via ultrasound imaging, visual observation, or tactile manipulation, and is intended to include at least the orbits, zygomatic process, coronoid process, condyles, external auditory meatus, coronal suture, the bregma, the pterion, the sagittal suture, the squamous suture, and the lambdoid sutures.

A “pressure” created by an ultrasound signal, as used herein, refers to a spatial peak pressure unless otherwise stated. It will be appreciated that a peak pressure can refer to either of the peak positive (compressional) or negative (rarefactional) pressure.

Systems and methods are provided herein for providing neuromodulation via focused ultrasound using a plurality of transducers having overlapping focal volumes. Effective, long-lasting therapeutic ultrasound treatment requires significant signal dosage within the treatment region, but many treatment targets can be adjacent to regions of the brain for which the application of energy may be undesirable. By employing multiple steerable transducers, each providing less than a desired signal dose within their focal volumes, the region receiving the desired signal dose can be restricted to a limited region of overlap between the focal volumes, allowing for precise application of neuromodulation to small target regions.

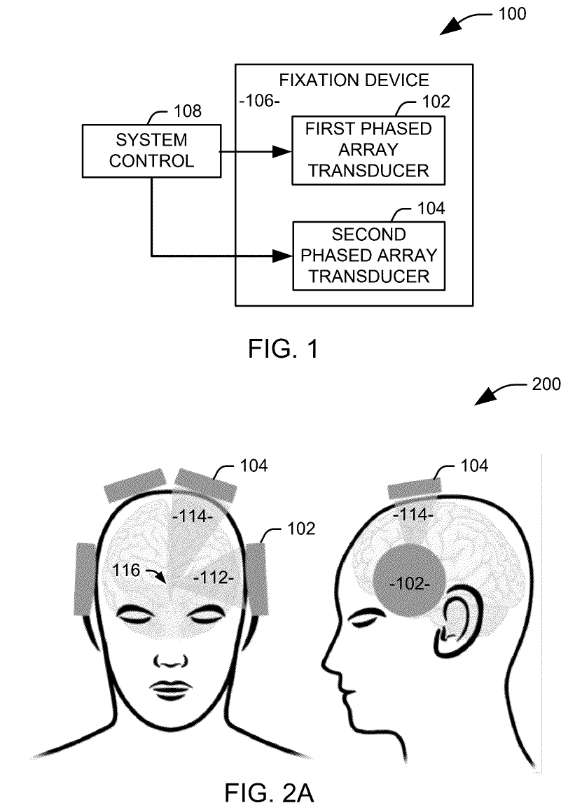

FIG. 1 illustrates a system 100 for providing neuromodulation via focused ultrasound. The system 100 includes first and second phased array ultrasound transducers 102 and 104 positioned within a fixation device 106 to provide ultrasound signals to a region of interest. In particular, the first phased array ultrasound transducer 102 can be positioned within the fixation device 106 to provide a first ultrasound beam through the temporal ultrasound window of a patient with a first focal volume within the region of interest. The second phased array ultrasound transducer 104 is positioned within the fixation device 106 to provide a second ultrasound beam through one of a frontal bone, a parietal bone, a sagittal suture, or a coronal suture of the patient with a second focal volume within a region of interest. The signal dose within each of the first focal volume and the second focal volume can be selected to be less than a threshold dose associated with therapeutic efficacy, with therapeutic efficacy achieved only in a region of overlap between the first focal volume and the second focal volume.

In one example, the threshold dose can be a threshold pressure, specifically a spatial peak pressure above one megapascal. In one implementation in accordance with this example, the threshold dose can be represented as a spatial peak pressure between one megapascal and 2.5 megapascal. In another example, the threshold pressure can be a spatial peak pressure above 0.3 megapascal. In one implementation in accordance with this example, the threshold dose can be represented as a spatial peak pressure between 0.3 megapascal and 2.5 megapascal. In a further example, the threshold dose can be represented as a spatial peak pressure of about two megapascals. Alternatively, the signal dose can be measured as a Mechanical Index, which is defined as the derated rarefactional pressure divided by the square root of the frequency in megahertz. In one example, the threshold dose can be a Mechanical Index between one and five. In another example, the threshold dose can be a Mechanical Index between two and five.

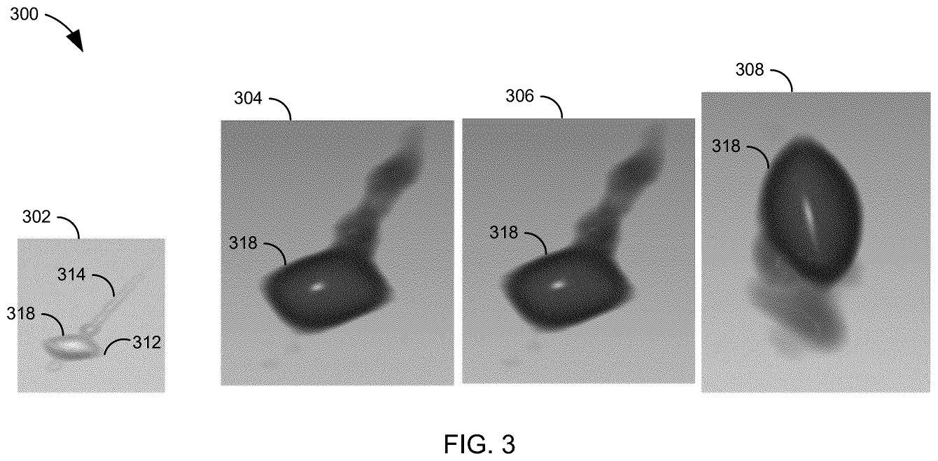

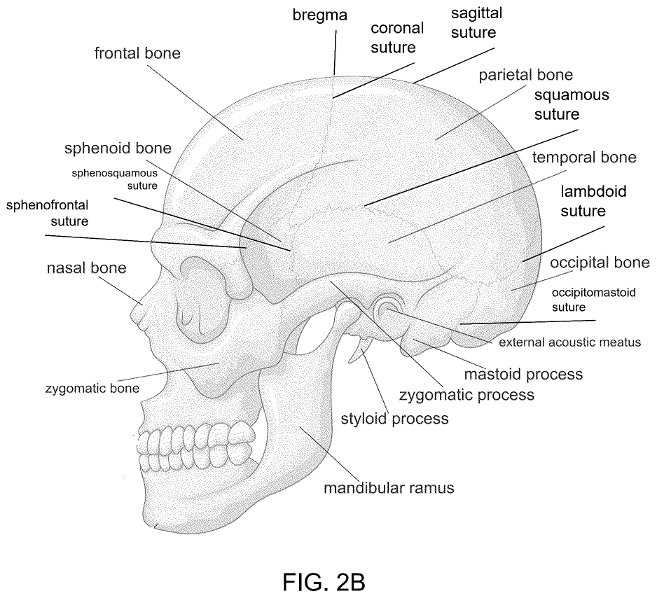

FIG. 2A is a diagram 200 illustrating an example placement of the first phased array ultrasound transducer 102 and the second phased array ultrasound transducer 104 of the system of FIG. 1. For example, each of the first and second phased array transducers 102 and 104 can be positioned relative to one or more skull landmarks. FIG. 2B illustrates the location of a number of skull landmarks that can be used for this purpose. In the illustrated example of FIG. 2A, the first phased array ultrasound transducer 102 is centered on or near the pterion, and the second phased array ultrasound transducer 104 is centered on or near the bregma. Each of a first ultrasound beam 112 from the first phased array ultrasound transducer 102 and a second ultrasound beam 114 from a second phased array ultrasound transducer 104 have focal regions within a region of interest 116 associated with a treatment. As stated previously, the dosage associated with the focal region of each beam is below the threshold dose outside of a region of overlap of the two beams 112 and 114 within the region of interest.

FIG. 3 illustrates a simulation 300 of the region of overlap 118 of the first 112 and second 114 ultrasound beams of FIG. 2 within the region of interest 116. A first image 302 illustrates the intersection of the first and second ultrasound beams 112 and 114. A width of the region of overlap 118 in a mediolateral view 304, and thus the region of tissue subjected to a therapeutic dosage, is 11.4 millimeters. In another example, this width is between nine and thirteen millimeters. A width of the region of overlap 118 in an inferosuperior view 306 is 5.7 millimeters. A width of the region of overlap 118 in an anterior-posterior view 308 is 6.1 millimeters. In another example, this width of the region of interest in each of the inferosuperior view 306 and the anterior-posterior view 308 is between four and eight FIG. 3 illustrates a simulation 300 of the region of overlap 118 of the first 112 and second 114 ultrasound beams within the region of interest 116. millimeters.

Returning to FIG. 1, the first phased array ultrasound transducer 102 can be configured as any two-dimensional array transducer capable of providing a focal region within the region of interest. In one example, the first phased array ultrasound transducer 102 is implemented as a rectangular array transducer. In another example, the first phased array ultrasound transducer is implemented as to have a cross-section that is substantially circularly symmetric along the axis of ultrasound propagation. In one example consistent with this implementation, the first phased array ultrasound transducer 102 can be implemented as an annular array, such as a segmented annular array.

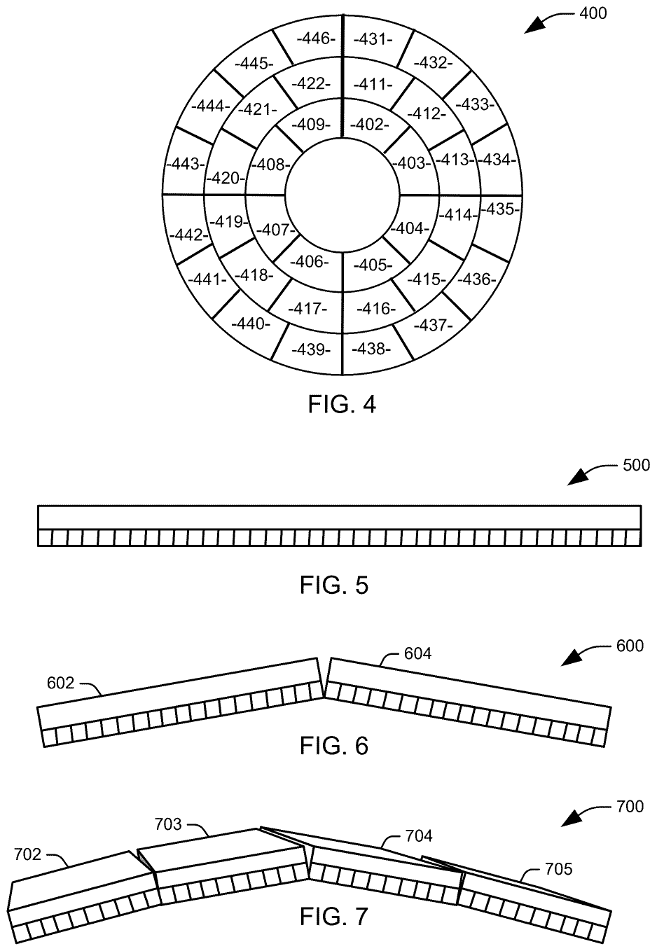

FIG. 4 illustrates one example of a segmented annular array transducer 400 that can be used with the system of FIG. 1 for either of the first phased array ultrasound transducer 102 or the second phased array ultrasound transducer 104. The illustrated array transducer 400 includes a plurality of transducer elements 402-409, 411-422, and 431-446 controlled by a system control (not shown) to provide an electrically steerable ultrasound beam. The plurality of transducer elements 402-409, 411-422, and 431-446 are arranged in three annular rings, with a first set of transducer elements 402-409 forming an innermost ring of the annular array transducer 400, a second set of transducer elements 411-422 forming an center ring of the annular array transducer, and a third set of transducer elements 431-446 forming an outermost ring of the annular array transducer 400 In one implementation, an amplitude and phase of the output of each of the plurality of transducer elements 402-409, 411-422, and 431-446 can be controlled by the system control to control a direction, focal depth, and intensity of the ultrasound beam provided by the segmented annular array transducer 400.

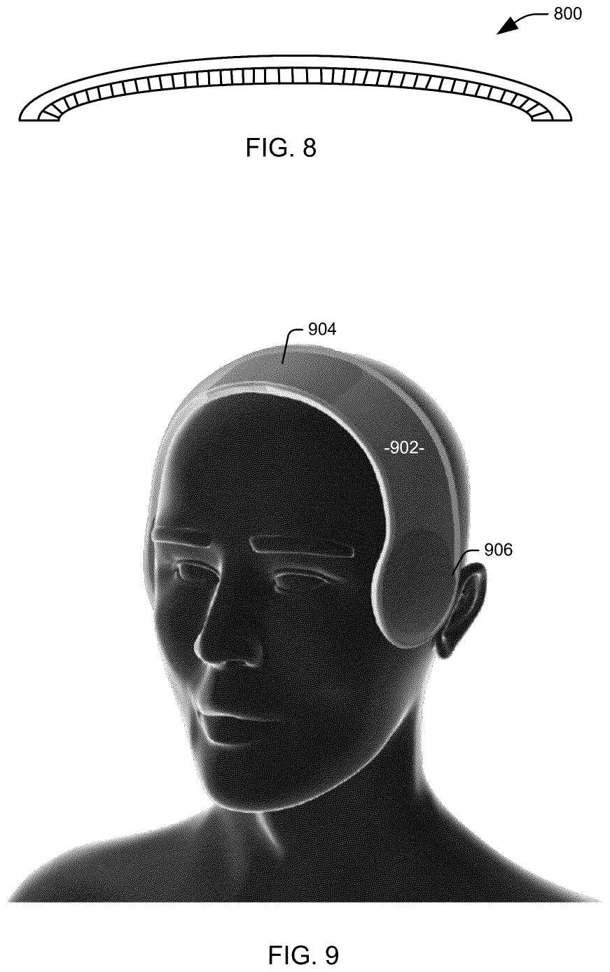

Returning to FIG. 1, the second phased array ultrasound transducer 104 can be implemented as any phased array transducer capable of providing a focal region within the region of interest. For example, the second phased array ultrasound transducer 104 can include any of a linear, rectangular, or annular array of elements. In one implementation, the second phased ultrasound transducer can be centered on or near a bregma of the patient. FIG. 5 illustrates one example of a rectangular array transducer 500, having one or more rows of elements, that can be used with the system of FIG. 1. FIG. 6 illustrates another example of a rectangular array transducer 600 implemented as a segmented rectangular array, having a plurality of rectangular sections 602 and 604, each having one or more rows of elements, that can be used with the system of FIG. 1. The two rectangular sections are positioned at an acute angle relative to one another. In one example, the angle is less than thirty degrees. FIG. 7 illustrates a further example of a rectangular array transducer 700 implemented as a segmented rectangular array, having a plurality of rectangular sections 702-705 each having one or more rows of elements, that can be used with the system of FIG. 1. The rectangular sections 702-705 are each positioned at an acute angle with a neighboring rectangular section. In one example, the acute angle can be less than thirty degrees. FIG. 8 illustrates a still further example of a rectangular array transducer 800, implemented as a curved linear array, that can be used with the system of FIG. 1.

FIG. 9 illustrates an example implementation of a wearable fixation device 902 that can be used with the system of FIG. 1. In the illustrated implementation, the fixation device 902 is implemented as a headset, but it will be appreciated that the fixation device can be any device that fits to a patient's head and which can be dynamically adjusted, for example, with cups, straps, curved form factors and other features, to allow for a reproducible and precise delivery with minimal movement. Another example of an appropriate fixation device could include a helmet or similar device that can be adjusted to fit a patient's head. The illustrated fixation device 902 includes a central portion 904 that holds the second transducer at a position near the top of the patient's head and a rounded end portion 906 that holds the first transducer near an ear of the patient.

In one implementation, the fixation device 902 can be adjustable to work for multiple patients in a clinical environment. For example, the rounded ear portion 906 holding the first transducer can be adjusted to provide an appropriate position and angle for the transducer in the region of the pterion to allow for sonication via the ultrasound temporal window of a patient. Similarly, the central portion 904 can be adjusted for the diameter of the head to maintain the second transducer near one of a frontal bone, a parietal bone, a sagittal suture, or a coronal suture of the patient. In another implementation, a personalized fixation device can be fabricated for the patient to their specific measurements. For example, the fixation device 902 can be fabricated by three-dimensional printing or another rapid prototyping technology. Physical fiducials can be used to properly align the device relative to landmarks on the exterior of the head. In the example of a helmet, an extension of the fixation device 902 can be configured to align with the bridge of the patient's nose. A personalized fixation device, along with the movement detection provided by the ultrasound transducers, can allow for use of the device outside of clinical settings. In this implementation, the system 900 may be configured to provide neuromodulation at lower doses than are generally used in clinical settings, possibly for longer treatment durations.

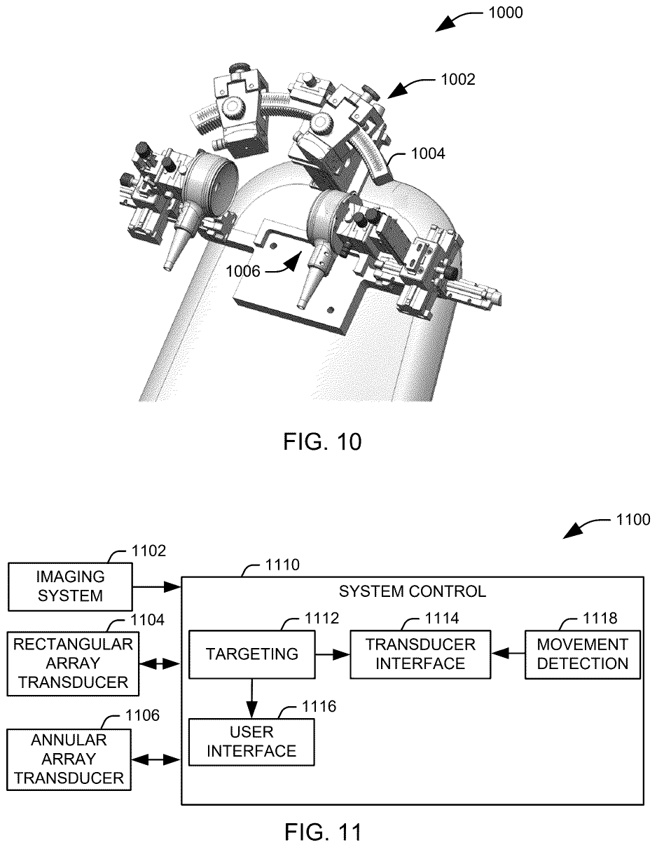

FIG. 10 illustrates another example implementation of a fixation device 1000 that can be used with the system of FIG. 1. In the illustrated implementation, the fixation device 1000 can be mounted to a chair or bed occupied by the patient. The illustrated fixation device 1000 includes a central portion 1002 that holds the second phased array ultrasound transducer at a position near the top of the patient's head. The central portion 1002 can be translated along a rail to allow for mechanical targeting of the ultrasound beam. A rounded end portion 1006 that holds the first array transducer near an ear of the patient. The fixation device 1000 can be adjustable to work for multiple patients in a clinical environment. For example, the rounded ear portion 1006 holding the first transducer can be adjusted to provide an appropriate position and angle for the transducer in the region of the pterion to allow for sonication via the ultrasound temporal window of a patient. Similarly, the central portion 1004 can be adjusted along the rail to maintain the second transducer in an appropriate position near one of a frontal bone, a parietal bone, a sagittal suture, or a coronal suture of the patient. Physical fiducials can be used to properly align the device relative to landmarks on the exterior of the head.

Returning to FIG. 1, a system control 108 controls each of the first phased array ultrasound transducer 102 and the second phased array ultrasound transducer 104 such that the first focal volume overlaps with the second focal volume as to collectively provide a signal dose within a region of overlap in a region of interest that is greater than the threshold dose. For example, the system control 108 can control each of the first phased array ultrasound transducer 102 and the second phased array ultrasound transducer to electrically steer the ultrasound beams provided by arrays of elements within each transducer to provide the region of overlap. In one example, the system control can control each transducer 102 and 104 as to adjust a position of the region of interest by at least three millimeters along each of a frontal axis, a sagittal axis, and a vertical axis. Accordingly, in the region of overlap, the dose of the combined signals is greater than or equal to the desired threshold dose. By carefully aligning the first focal volume and the second focal volume, the volume of the region of overlap can be significantly smaller than the first focal volume and the second focal volume, allowing the region in which the combined signal dose exceeds the desired threshold to be limited to a small space within the region of interest. It will be appreciated that this alignment can include one or both physical alignment of the transducers relative to the region of interest, for example, using an adjustable fixation device 106 and electronic steering of the transducers 102 and 104, for example, by implementing the transducers as array transducers. As a result, neuromodulation can be applied with significant precision, allowing for avoidance of surrounding tissue for which application of ultrasound energy would be undesirable due to the potential for creating side effects. In one example, the first phased array ultrasound transducer 102 and the second phased array ultrasound transducer 104 can be aligned such that a longest principal axis of the first focal volume forms an oblique angle with a longest principal axis of the second focal volume. In another example, the first ultrasound transducer 102 and the second phased array ultrasound transducer 104 can be aligned such that the longest principal axis of the first focal volume is substantially orthogonal to a longest principal axis of the second focal volume. In one example, the system control 108 can control the frequencies of the signals produced first and second phased array ultrasound transducers to maintain a desired ratio between their frequencies. In one example, a frequency ratio of between 1.1 and 1.4 to 1 is maintained. In another implementation, a frequency ratio of about 1.25 to 1 is maintained. In combination with the oblique angle of the two ultrasound beams, this frequency ratio allows for a smoothing of pressure peaks within the region of overlap, allowing for a more consistent pressure within the region.

In addition, the system control 108 can control relative magnitudes of the first ultrasound signal and the second ultrasound signal to control a directionality of the energy provided within the region of overlap. In particular, the relative magnitudes of the first ultrasound signal and the second ultrasound signal can be controlled such that an Umov-Poynting vector of the ultrasound energy within the region of overlap aligns in a desired direction. In one implementation, a direction of the nervous system tissue within the region of overlap can be determined from previous imaging, and the relative magnitudes of the first ultrasound signal and the second ultrasound signal can be controlled to substantially align the Umov-Poynting vector in a direction parallel with the direction of the nervous system tissue. In another implementation, the relative magnitudes of the first ultrasound signal and the second ultrasound signal can be controlled to substantially align the Umov-Poynting vector in a direction perpendicular to the direction of the nervous system tissue.

The system 100 allows for precise targeting of deep brain targets, such as, for example, the nucleus accumbens, the sensory thalamus, the anterior limb of the inferior colliculus, the subthalamic nucleus, the globus pallidus internus, the periventricular grey matter, the periaqueductal grey matter, the pedunculopontine nucleus, the ventral intermediate nucleus, the ventral capsule, the ventral striatum the thalamic nuclei pulvinar nucleus, the caudate body, the anterior cingulate cortex, the subgenual cingulate cortex, the nucleus basalis of Meynert, the anterior insula, the posterior insula, the cingulum, and the amygdala. By limiting the region of therapeutic efficacy to the intersection of the focal volume of the circularly symmetric beam from the first phased array ultrasound transducer 102 and focal volume of the beam from the second phased array ultrasound transducer 104, these structures can be targeted with a volume significantly less than a cubic centimeter. In one example, it will be appreciated that the size and shape of the area of overlap can be tailored to a desired target region.

FIG. 11 illustrates an example implementation of a system 1100 for providing neuromodulation via focused ultrasound. The system 1100 includes an imaging system 1102 that obtains an image of a region of interest within the brain. It will be appreciated that the imaging can be performed prior to the procedure or during the procedure. The imaging system 1102 can include any appropriate imaging modality for determining a structure of the brain, including magnetic resonance imaging (MRI) imaging, computed tomography (CT) imaging, or ultrasound imaging. Where ultrasound imaging is used intraprocedurally, it will be appreciated that the imaging system 1102 can utilize either or both of a rectangular array transducer 1104 and an annular array transducer 1106 as one or both of a transmitter and a receiver to perform the imaging, for example, using an echo-pulse imaging approach. It will be appreciated that multiple imaging modalities can be employed. In one implementation, each of a MRI image, one or both of a CT scan and an X-ray of the skull, and ultrasound imaging can be used, with the MRI image and the one of the CT scan and the X-ray acquired before the procedure and the ultrasound imaging performed intraprocedurally.

In one implementation, a third transducer (not shown) can also be used, for example, on an opposite side of the head from the first transducer 102 to facilitate additional imaging and determine effects of the skull and tissue on ultrasound signal propagation. In one example, the first 102 and third transducers can be implemented as array transducers and each element of the array can be activated in sequence, with the signal received at the opposing transducer array, to evaluate how the signal produced at each element propagates through the tissue. During this process, as well as any imaging performed via ultrasound, multiple signal frequencies can be used to evaluate the effects of the skull and tissue along the path of the beam on the signal.

The image of the brain and any additional information collected at the imaging system 1102 can be provided to a system control 1110. A targeting component 1112 determines appropriate settings for the rectangular transducer 1104 and the annular transducer 1106. It will be appreciated that the system control 1110 can be implemented as machine-readable instructions executed by an associated processor, as dedicated hardware, or as a combination of dedicated hardware and software. Similarly, any data on ultrasound signal propagation can be used in refining the settings for the rectangular transducer 1104 and the annular transducer 1106. It will be appreciated that the image can be used both to find a target within the brain as well as to find one or more anatomical landmarks to position the linear transducer 1104 and the annular transducer 1106. For example, the linear transducer 1104 may be positioned at or near the coronal suture, and the image can be used to locate the coronal suture. In one implementation, ultrasound imaging is used to locate each of the coronal, sagittal, squamous, and lambdoid sutures to guide the placement of the transducers 1104 and 1106.

During operation, the rectangular transducer 1104 is positioned near a coronal suture of the patient to provide an ultrasound beam that travels generally in an inferior direction into the brain. In one implementation, the transducer is implemented as a rectangular curved array of elements and is utilized to follow the contour of the skull, such that each element of the array can be placed approximately parallel to the skull to minimize shear waves, which can distort the ultrasound beam. In another implementation, the curved array may be approximated by a series of rectangular array transducers positioned end to end and angled with respect to one another. The annular transducer 1106 can be placed on or near a pterion of the user to provide an ultrasound beam that travels medially to intersect the ultrasound beam provided by the rectangular transducer 1104. In one example, the annular transducer 1106 is implemented as a segmented annular array of elements to allow for additional capability in electronic steering to compensate for non-uniform bone effects and add additional range for the beam in the sagittal plane. It will be appreciated that the system control 1110, via a transducer interface 1114, instructs each transducer 1104 and 1106 to maintain the intensity of each beam below a threshold dose associated with therapeutic efficacy, while ensuring that the region of intersection of the two beams provides a combined signal dose above the threshold for therapeutic efficacy.

In one example, the targeting component 1112 receives a desired target location within the brain, selected by a user, for example, on an image provided by the imaging system 1102, and projected locations for the rectangular array transducer 1104 and an annular array transducer 1106 and determines appropriate settings for the two transducers. In another implementation, the targeting component 1112 receives the desired target location and determines both appropriate locations and settings for the rectangular array transducer 1104 and an annular array transducer 1106, with the locations selected from a range of available locations for the arrays. For example, the rectangular array transducer 1104 can be placed within a range of locations around the coronal suture or bregma and the annular array transducer 1106 can be placed on or near one the pterion. Where two annular array transducers are used, an annular array transducer can be placed on or near each pterion. The selected locations can be provided to the user at a user interface 1116.

The targeting component 1112 can select settings for the transducers 1104 and 1106 as to provide ultrasound beams that overlap at the desired target, with the settings provided to the transducers 1104 and 1106 via the transducer interface 1114. The transducer interface 1114 can include both appropriate software for interfacing with the transducers as well as a data connection to the transducers. It will be appreciated that this determination can be informed by information received at the imaging system 1102, such as a thickness of the skull at the locations of the rectangular array transducer 1104 and the annular array transducer 1106, an estimated density of the tissue along the path of the beam, as estimated by computed tomography imaging, and any additional information on ultrasound propagation determined at the imaging component 1102. For example, a number of internal brain landmarks can be identified by the targeting component 1110 from an MRI image and used to locate the target. In one implementation, the internal landmarks can include the anterior commissure, the posterior commissure, and the middle cerebellar peduncle. To further restrict the treatment volume, the targeting component 1112 can instruct the two arrays to operate at different frequencies, as to produce a desired interference pattern within the region of overlap between the focal volumes of the two beams. In this instance, the intensity of the beams can be selected such that a signal dose above a threshold dose associated with therapeutic efficacy is achieved only at locations of constructive interference between the two beams. Alternatively, the different frequencies can be selected to provide a substantially uniform dose across the region of overlap, for example, by maintaining a ratio of about 1.25 to 1.

Further, the targeting component 1110 can instruct the transducer interface 1116 control relative intensities of the beams provided by the two transducers 1104 and 1106, so long as a combined intensity of the beams within a region of interest associated with the target is sufficient to provide a threshold dose associated with therapeutic efficacy. In one example, the targeting component 1110 can evaluate the projected path of each beam for portions of the brain for which application of the ultrasound energy would be particularly likely to cause undesirable side effects. Where a given beam is projected to pass through or near such a region, the transducer associated with that beam can be controlled to reduce the intensity of the beam and the other transducer can be controlled to increase the intensity of the other beam to maintain the combined intensity of the beams to provide the threshold dose at the target while decreasing the energy delivered to portions of the brain associated with side effects. Alternatively or additionally, the relative magnitudes of the provided by the two transducers 1104 and 1106 can be controlled such that an Umov-Poynting vector of the ultrasound energy at the target aligns in a desired direction, for example, a direction associated with a contour of the nervous system tissue.

A movement detection component 1118 ensures that the transducer assemblies remain in position during treatment, both to maintain the dose at the target and to avoid providing energy to portions of the brain associated with negative side effects. In some implementations, neuromodulation treatment can last twenty minutes or more, and movement of the patient can result in displacement of the transducers 1104 and 1106 relative to the skull. To detect this movement, ultrasound imaging can be performed using one or more ultrasound transducers, which can include the transducers 1104 and 1106, to detect skull landmarks, including any of the orbits, zygomatic process, coronoid process, condyles, external auditory meatus, coronal suture, the bregma, the pterion, the sagittal suture, the squamous suture, and the lambdoid sutures, and the position of the transducers relative to these landmarks can be compared to the original positions of the transducers. In some implementations, the transducers can use infrared imaging for this purpose in place of ultrasound imaging. In one example, the arrays 1104 and 1106 can be configured to give an audible beep or visible signal to the user when a transducer is not in the original location and will alert until it is moved to the appropriate location.

The system 1100 provides superior focal characteristics with minimal added complexity. The annular array transducer 1106 provides an ellipsoidal beam with a small circular cross section, with a long axial extent, and is suitable for use on the side of the head, where it needs to be situated in the limited space allowed given the ears and the jaw of the patient. The rectangular array 1104 provides a fan-shaped beam that can be controlled such that the region of overlap is well defined as a slice of the ellipsoidal focal region provided by the annular array 1106. Having electronic control of both arrays 1104 and 1106 via the system control 1110 allows for positioning control and refinement and provides the capacity for modulating multiple targets. For instance, the beam from the annular array 1106 can extend across the brain to hit bilateral targets simultaneously, and the rectangular transducer 1104 can provide a beam from the top of the head to switch quickly between the two targets, or potentially, sonicate them both simultaneously, with the rectangular array creating two separate beams.

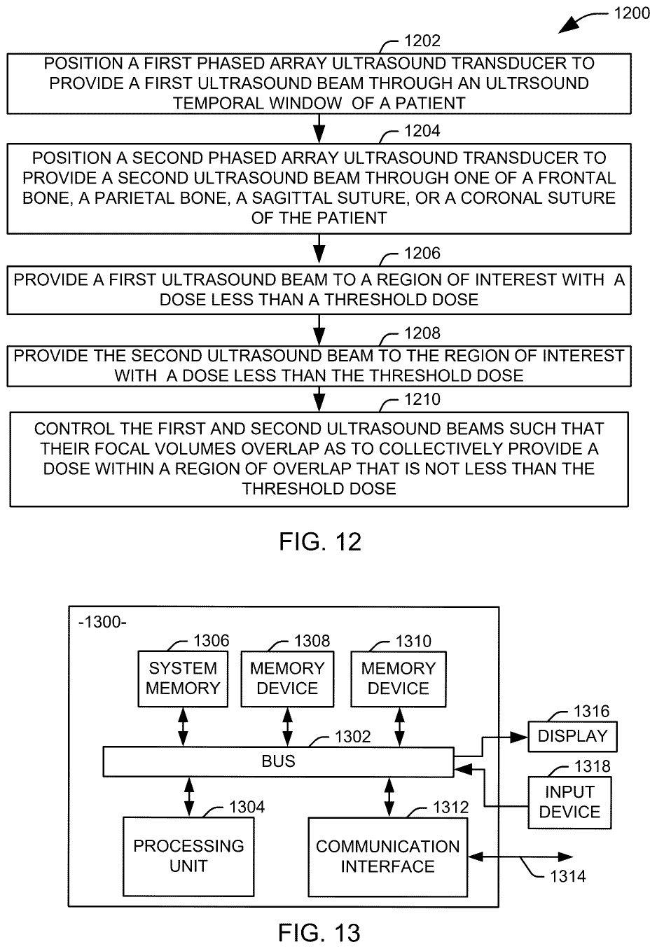

In view of the foregoing structural and functional features described above, an example method will be better appreciated with reference to FIG. 12. While, for purposes of simplicity of explanation, the example method of FIG. 12 is shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement a method in accordance with the invention.

FIG. 12 illustrates one example of a method 1200 for providing neuromodulation via focused ultrasound. At 1202, a first phased array ultrasound transducer is positioned such that a first ultrasound beam provided by the first phased array ultrasound transducer will pass through the temporal ultrasound window of a patient. At 1204, a second phased array ultrasound transducer is positioned such that a second ultrasound beam provided by the second phased array ultrasound transducer will pass through one of a frontal bone, a parietal bone, a sagittal suture, or a coronal suture of the patient. At 1206, the first ultrasound beam is provided with a first focal volume within a region of interest, with a signal dose within the first focal volume that is less than a threshold dose associated with therapeutic efficacy. At 1208, the second ultrasound beam is provided with a second focal volume within the region of interest, with a signal dose within the second focal volume being less than the threshold dose. In one example, the first ultrasound beam and the second ultrasound beam are provided such that the longest principal axis of the first focal volume forms an oblique angle with the longest principal axis of the second focal volume.

At 1210, each of the first ultrasound beam and the second ultrasound beam are controlled such that the first focal volume overlaps with the second focal volume as to collectively provide a signal dose within a region of overlap in the region of interest that is greater than the threshold dose. For example, the beams can be controlled by electronic steering of the array to create the region of overlap. In one example, the relative intensity of the first ultrasound beam and the second ultrasound beam can also be controlled as to align a Umov-Poynting vector of ultrasound energy within the region of overlap in a desired direction, for example, to align with a direction of nervous system tissue in the region of interest.

FIG. 13 is a schematic block diagram illustrating an exemplary system 1300 of hardware components capable of implementing examples of the systems and methods disclosed in FIGS. 1-12, such as the control system illustrated in FIGS. 1 and 11. The system 1300 can include various systems and subsystems. The system 1300 can be any of a personal computer, a laptop computer, a workstation, a computer system, an appliance, an application-specific integrated circuit (ASIC), a server, a server blade center, or a server farm.

The system 1300 can includes a system bus 1302, a processing unit 1304, a system memory 1306, memory devices 1308 and 1310, a communication interface 1312 (e.g., a network interface), a communication link 1314, a display 1316 (e.g., a video screen), and an input device 1318 (e.g., a keyboard and/or a mouse). The system bus 1302 can be in communication with the processing unit 1304 and the system memory 1306. The additional memory devices 1308 and 1310, such as a hard disk drive, server, stand-alone database, or other non-volatile memory, can also be in communication with the system bus 1302. The system bus 1302 interconnects the processing unit 1304, the memory devices 1306-1310, the communication interface 1312, the display 1316, and the input device 1318. In some examples, the system bus 1302 also interconnects an additional port (not shown), such as a universal serial bus (USB) port.

The system 1300 could be implemented in a computing cloud. In such a situation, features of the system 1300, such as the processing unit 1304, the communication interface 1312, and the memory devices 1308 and 1310 could be representative of a single instance of hardware or multiple instances of hardware with applications executing across the multiple of instances (i.e., distributed) of hardware (e.g., computers, routers, memory, processors, or a combination thereof). Alternatively, the system 1300 could be implemented on a single dedicated server.

The processing unit 1304 can be a computing device and can include an application-specific integrated circuit (ASIC). The processing unit 1304 executes a set of instructions to implement the operations of examples disclosed herein. The processing unit can include a processing core.

The additional memory devices 1306, 1308, and 1310 can store data, programs, instructions, database queries in text or compiled form, and any other information that can be needed to operate a computer. The memories 1306, 1308 and 1310 can be implemented as computer-readable media (integrated or removable) such as a memory card, disk drive, compact disk (CD), or server accessible over a network. In certain examples, the memories 1306, 1308 and 1310 can comprise text, images, video, and/or audio, portions of which can be available in formats comprehensible to human beings.

Additionally or alternatively, the system 1300 can access an external data source or query source through the communication interface 1312, which can communicate with the system bus 1302 and the communication link 1314.

In operation, the system 1300 can be used to implement one or more parts of a focused ultrasound neuromodulation system in accordance with the present invention. Computer executable logic for implementing the system control for an ultrasound neuromodulation system resides on one or more of the system memory 1306, and the memory devices 1308, 1310 in accordance with certain examples. The processing unit 1304 executes one or more computer executable instructions originating from the system memory 1306 and the memory devices 1308 and 1310. It will be appreciated that a computer readable medium can include multiple computer readable media each operatively connected to the processing unit.

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software code can be stored in a memory. Memory can be implemented within the processor or external to the processor. As used herein the term “memory” refers to any type of long term, short term, and volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine readable mediums for storing information. The terms “computer readable medium” and “machine readable medium” includes, but is not limited to portable or fixed storage devices, optical storage devices, wireless channels, and/or various other storage mediums capable of storing that contain or carry instruction(s) and/or data. It will be appreciated that a “computer readable medium” or “machine readable medium” can include multiple media each operatively connected to a processing unit.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

1. A system comprising:
a fixation device;
a first phased array ultrasound transducer positioned within the fixation device to provide a first ultrasound beam through a temporal ultrasound window of a patient with a first focal volume within a region of interest, a signal dose within the first focal volume being less than a threshold dose associated with therapeutic efficacy;
a second phased array ultrasound transducer that is positioned within the fixation device to provide a second ultrasound beam through one of a frontal bone, a parietal bone, a sagittal suture, or a coronal suture of the patient with a second focal volume within a region of interest, a signal dose within the second focal volume being less than the threshold dose; and
a system control that controls each of the first phased array ultrasound transducer and the second phased array ultrasound transducer such that the first focal volume overlaps with the second focal volume as to collectively provide a signal dose within a region of overlap in the region of interest that is not less than the threshold dose.

2. The system of claim 1, wherein the first phased array ultrasound transducer is implemented as an annular array.

3. The system of claim 2, wherein the first phased array ultrasound transducer is implemented as a segmented annular array.

4. The system of claim 2, wherein the first phased array ultrasound transducer is implemented as a rectangular array.

5. The system of claim 1, wherein the second phased array ultrasound transducer is implemented as a rectangular array.

6. The system of claim 1, wherein the second phased array ultrasound transducer is implemented as a segmented rectangular array having a plurality of rectangular sections with each of the plurality of rectangular sections positioned at an acute angle with a neighboring rectangular section of the plurality of rectangular sections.

7. The system of claim 1, wherein a longest principal axis of the first focal volume is substantially orthogonal to a longest principal axis of the second focal volume.

8. The system of claim 1, wherein a longest principal axis of the first focal volume forms an oblique angle with a longest principal axis of the second focal volume.

9. The system of claim 1, wherein the system control controls each of the first phased array ultrasound transducer and the second phased array ultrasound transducer as to electronically alter respective positions of the first and second focal volumes.

10. The system of claim 6, wherein the system control controls each of the first phased array ultrasound transducer and the second phased array ultrasound transducer as to adjust a position of the region of interest by at least three millimeters along each of a frontal axis, a sagittal axis, and a vertical axis.

11. The system of claim 1, wherein the system control controls the relative intensity of the first ultrasound beam and the second ultrasound beam as to align a Umov-Poynting vector of ultrasound energy within the region of overlap in a desired direction.

12. The system of claim 1, the system control further comprising a targeting component that determines appropriate settings for the first phased array ultrasound transducer and the second phased array ultrasound transducer from an image provided to the system control.

13. The system of claim 12, wherein the image is produced via any of magnetic resonance imaging, computed tomography. Positron emission tomography, and single-photon emission computed tomography.

14. The system of claim 12, wherein the image is produced using one or both of the first phased array ultrasound transducer and the second phased array ultrasound transducer.

15. The system of claim 1, wherein the signal dose is measured as a spatial peak pressure, and the threshold dose is a spatial peak pressure between 0.3 megapascal and 2.5 megapascals.

16. The system of claim 1, the system control further comprising a movement detection component that receives ultrasound imaging data, detects skull landmarks within the ultrasound imaging data, and compares the position of the first and second ultrasound from an image provided to the system control.

17. A method comprising:
positioning a first phased array ultrasound transducer such that a first ultrasound beam provided by the first phased array ultrasound transducer will pass through a temporal ultrasound window of a patient; and
positioning a second phased array ultrasound transducer such that a second ultrasound beam provided by the second phased array ultrasound transducer will pass through one of a frontal bone, a parietal bone, a sagittal suture, or a coronal suture of the patient;
providing the first ultrasound beam with a first focal volume within a region of interest, a signal dose within the first focal volume being less than a threshold dose associated with therapeutic efficacy;
providing the second ultrasound beam with a second focal volume within the region of interest, a signal dose within the second focal volume being less than the threshold dose; and
controlling each of the first ultrasound beam and the second ultrasound beam such that the first focal volume overlaps with the second focal volume as to collectively provide a signal dose within a region of overlap in the region of interest that is not less than the threshold dose.

18. The method of claim 17, further comprising providing the first ultrasound beam and the second ultrasound beam such that a longest principal axis of the first focal volume is substantially orthogonal to a longest principal axis of the second focal volume.

19. The method of claim 17, wherein controlling each of the first ultrasound beam and the second ultrasound beam comprises controlling the relative intensity of the first ultrasound beam and the second ultrasound beam as to align a Umov-Poynting vector of ultrasound energy within the region of overlap in a desired direction.

20. A system comprising:
a fixation device;
a segmented annular array ultrasound transducer positioned within the fixation device to provide a first ultrasound beam through a temporal ultrasound window of the patient with a first focal volume within a region of interest, a signal dose within the first focal volume being less than a threshold dose associated with therapeutic efficacy;
a rectangular array ultrasound transducer that is positioned within the fixation device to provide a second ultrasound beam through one of the frontal bone, the parietal bone, a sagittal suture, or the coronal suture with a second focal volume within a region of interest, a signal dose within the second focal volume being less than the threshold dose; and
a system control that controls each of the first array ultrasound transducer and the second array ultrasound transducer such that the first focal volume overlaps with the second focal volume as to collectively provide a signal dose within a region of overlap in the region of interest that not less than the threshold dose.

21. The system of claim 20, wherein the rectangular array transducer is implemented as a segmented rectangular array having a plurality of rectangular sections with each of the plurality of rectangular sections positioned at an acute angle with a neighboring rectangular section of the plurality of rectangular sections.
